SEQUENCE LISTING (200 sequences)

SEQ ID NO: 1 - protein, 15 aa, Artificial Sequence
  derived from LipC
DLLTPGINEVRRRDR

SEQ ID NO: 2 - protein, 15 aa, Artificial Sequence
  derived from LipC
RNAPPFLVIHGSRDC

SEQ ID NO: 3 - protein, 15 aa, Artificial Sequence
  derived from LipM
GGTAKTPGPLRMLRI

SEQ ID NO: 4 - protein, 15 aa, Artificial Sequence
  derived from Rv2349c
TPLTAPEGTPGEWIP

SEQ ID NO: 5 - protein, 15 aa, Artificial Sequence
  derived from LipG
KTLAVIFSSNNHRFL

SEQ ID NO: 6 - protein, 15 aa, Artificial Sequence
  derived from LipO
FTTDAPGRREFVGLL

SEQ ID NO: 7 - protein, 15 aa, Artificial Sequence
  derived from Rv2301
TAACPDAEVVFARGR

SEQ ID NO: 8 - protein, 15 aa, Artificial Sequence
  derived from Rv1984
QRTVASCPNTRIVLG

SEQ ID NO: 9 - protein, 15 aa, Artificial Sequence
  derived from Rv3452
CNNGDPICSDGNRWR

SEQ ID NO: 10 - protein, 15 aa, Artificial Sequence
  derived from Rv3452
MIPRPQPHSGRWRAG

SEQ ID NO: 11 - protein, 15 aa, Artificial Sequence
  derived from Rv2351c
AGVTIPFRLDTTRGP

SEQ ID NO: 12 - protein, 16 aa, Artificial Sequence
  derived from LipT
TRAVLVFDRRCRIEFD

SEQ ID NO: 13 - protein, 15 aa, Artificial Sequence
  derived from LipW
GYVMGTAQQDDRLCL

SEQ ID NO: 14 - protein, 15 aa, Artificial Sequence
  derived from LipW
DDRPSIAPANPHYRL

SEQ ID NO: 15 - protein, 15 aa, Artificial Sequence
  derived from LipW
DADARVAVPGRRDDL

SEQ ID NO: 16 - protein, 15 aa, Artificial Sequence
  derived from Rv0183
MPRRAPALTAPLLVL

SEQ ID NO: 17 - protein, 15 aa, Artificial Sequence
  derived from LipC
ASDFLSATAKDLLTP

SEQ ID NO: 18 - protein, 15 aa, Artificial Sequence
  derived from LipC
VDKFGGDRNFIAVAG

SEQ ID NO: 19 - protein, 15 aa, Artificial Sequence
  derived from Rv1984
HADPCSDIAVVFARG

SEQ ID NO: 20 - protein, 15 aa, Artificial Sequence
  derived from LipC
QLLDVWRRKDMPTKP

SEQ ID NO: 21 - protein, 15 aa, Artificial Sequence
  derived from LipC
HLSALAGLTANDPQY

SEQ ID NO: 22 - protein, 15 aa, Artificial Sequence
  derived from Rv3452
AIALFGNPSGRAGGL

SEQ ID NO: 23 - protein, 15 aa, Artificial Sequence
  derived from Rv2301
NFSPAYNDRTIELCH

SEQ ID NO: 24 - protein, 15 aa, Artificial Sequence
  derived from LipF
VRAPGVRAADGAGRV

SEQ ID NO: 25 - protein, 15 aa, Artificial Sequence
  derived from Rv0183
RLIPIEGSRRLVECV

SEQ ID NO: 26 - protein, 15 aa, Artificial Sequence
  derived from LipG
AKGLRVIRYDNRDVG

SEQ ID NO: 27 - protein, 15 aa, Artificial Sequence
  derived from Rv2351c
DENGGFFDHVTPPTA

SEQ ID NO: 28 - protein, 12 aa, Artificial Sequence
  derived from Rv2350c
TAPTRGIPSGLC

SEQ ID NO: 29 - protein, 15 aa, Artificial Sequence
  derived from LipT
RVSNEVQRRWRCFSQ

SEQ ID NO: 30 - protein, 15 aa, Artificial Sequence
  derived from LipC
DRSTPERARFVDFLE

SEQ ID NO: 31 - protein, 15 aa, Artificial sequence
  derived from Rv0183
MWAEKSPRRSSAGSR

SEQ ID NO: 32 - protein, 15 aa, Artificial sequence
  derived from Rv0183
LVECVGSADVQLKEY

SEQ ID NO: 33 - protein, 15 aa, Artificial sequence
  derived from LipC
MNQRRAAGSTGVAYI

SEQ ID NO: 34 - protein, 15 aa, Artificial sequence
  derived from LipC
RTIDRHPEVFRDASP

SEQ ID NO: 35 - protein, 15 aa, Artificial sequence
  derived from LipC
APERTPPVCGALRHR

SEQ ID NO: 36 - protein, 15 aa, Artificial sequence
  derived from LipC
ELPGAGHGFDLLDGA

SEQ ID NO: 37 - protein, 15 aa, Artificial sequence
  derived from LipC
SPDDLAVEWPAPERT

SEQ ID NO: 38 - protein, 15 aa, Artificial sequence
  derived from LipC
ALRHRRYVHRRRVLY

SEQ ID NO: 39 - protein, 15 aa, Artificial sequence
  derived from LipC
GSRDCVIPVEQARSF

SEQ ID NO: 40 - protein, 15 aa, Artificial sequence
  derived from LipC
EGSDTSVDAVVGIYG

SEQ ID NO: 41 - protein, 15 aa, Artificial sequence
  derived from Rv3452
PANGDFLAAADGAND

SEQ ID NO: 42 - protein, 15 aa, Artificial sequence
  derived from Rv1984
NGSDDASAHIQRTVA

SEQ ID NO: 43 - protein, 17 aa, Artificial sequence
  derived from Rv1984
MTSQAATFAANRLDHAG

SEQ ID NO: 44 - protein, 15 aa, Artificial sequence
  derived from Rv1984
VFARGTHQASGLGDV

SEQ ID NO: 45 - protein, 15 aa, Artificial sequence
  derived from LipF
PNIGTDAMFPARAFD

SEQ ID NO: 46 - protein, 15 aa, Artificial sequence
  derived from LipI
MPSLDNTADEKPAID

SEQ ID NO: 47 - protein, 15 aa, Artificial sequence
  derived from Rv3802c
SCPDVQMISVPGTWE

SEQ ID NO: 48 - protein, 15 aa, Artificial sequence
  derived from LipO
ATLPTEPMRSRGRNL

SEQ ID NO: 49 - protein, 15 aa, Artificial sequence
  derived from LipO
TPNDPRFQPGFEQVD

SEQ ID NO: 50 - protein, 12 aa, Artificial sequence
  derived from Rv2351c
TPVRGTPSGLCS

SEQ ID NO: 51 - protein, 15 aa, Artificial sequence
  derived from Rv2351c
TTRGPFLDGECVNDP

SEQ ID NO: 52 - protein, 15 aa, Artificial sequence
  derived from LipQ
ICVSINYSKSPRCTW

SEQ ID NO: 53 - protein, 15 aa, Artificial sequence
  derived from LipQ
GEKDPMVPSAQSRAF

SEQ ID NO: 54 - protein, 15 aa, Artificial sequence
  derived from LipQ
IYGRRMGARKGSLAL

SEQ ID NO: 55 - protein, 15 aa, Artificial sequence
  derived from LipQ
ENLLDIWRRPDLAPG

SEQ ID NO: 56 - protein, 15 aa, Artificial sequence
  derived from LipQ
SEAPPFFVLHGEKDP

SEQ ID NO: 57 - protein, 15 aa, Artificial sequence
  derived from LipR
EEMAAVYTRLLDDGL

SEQ ID NO: 58 - protein, 15 aa, Artificial sequence
  derived from Rv2350c
FKQAADPRSNLARFG

SEQ ID NO: 59 - protein, 15 aa, Artificial sequence
  derived from Rv2350c
DPAGVTLPYRFDTTR

SEQ ID NO: 60 - protein, 15 aa, Artificial sequence
  derived from Rv2350c
PLDFAADVRNNRLPK

SEQ ID NO: 61 - protein, 15 aa, Artificial sequence
  derived from Rv2350c
FDTTRGPLVAGECVN

SEQ ID NO: 62 - protein, 15 aa, Artificial sequence
  derived from LipT
IYRTRFGALLTAAAD

SEQ ID NO: 63 - protein, 15 aa, Artificial sequence
  derived from LipT
DGVHRWRSIPYARAP

SEQ ID NO: 64 - protein, 15 aa, Artificial sequence
  derived from LipT
RCFSQIGVPGDDWPA

SEQ ID NO: 65 - protein, 15 aa, Artificial sequence
  derived from LipU
GGAFLTCGANSHGRL

SEQ ID NO: 66 - protein, 10 aa, Artificial sequence
  derived from Rv1755c
PTRGIPSGPC

SEQ ID NO: 67 - protein, 15 aa, Artificial sequence
  derived from LipY
SVVQITPAHPTGEYV

SEQ ID NO: 68 - protein, 15 aa, Artificial sequence
  derived from Rv0183
FAYGVERPDNYDLMV

SEQ ID NO: 69 - protein, 15 aa, Artificial sequence
  derived from Rv0183
DFDTLVGIATREYPG

SEQ ID NO: 70 - protein, 15 aa, Artificial sequence
  derived from Rv0183
HGLGEHARRYDHVAQ

SEQ ID NO: 71 - protein, 15 aa, Artificial sequence
  derived from Rv0183
GRALLQVGETMPRRA

SEQ ID NO: 72 - protein, 15 aa, Artificial sequence
  derived from Rv0183
SAGSRPEFSASTLTS

SEQ ID NO: 73 - protein, 15 aa, Artificial sequence
  derived from Rv0183
EVFNEPERNQVLDDV

SEQ ID NO: 74 - protein, 15 aa, Artificial sequence
  derived from Rv0183
REYPGCKRIVLGHSM

SEQ ID NO: 75 - protein, 15 aa, Artificial sequence
  derived from Rv0183
RIVYDVWTPDTAPQA

SEQ ID NO: 76 - protein, 15 aa, Artificial sequence
  derived from Rv0183
DFTAISRDPEVVQAY

SEQ ID NO: 77 - protein, 15 aa, Artificial sequence
  derived from Rv0183
LVRDISEYTADFDTL

SEQ ID NO: 78 - protein, 15 aa, Artificial sequence
  derived from Rv0183
PLLVLHGTDDRLIPI

SEQ ID NO: 79 - protein, 15 aa, Artificial sequence
  derived from Rv0183
GLVTYALDHRGHGRS

SEQ ID NO: 80 - protein, 15 aa, Artificial sequence
  derived from Rv0183
GHGRSGGKRVLVRDI

SEQ ID NO: 81 - protein, 15 aa, Artificial sequence
  derived from LipC
QARSFVERLRAVSRS

SEQ ID NO: 82 - protein, 15 aa, Artificial sequence
  derived from LipC
RRRDRASTQEVSVAA

SEQ ID NO: 83 - protein, 15 aa, Artificial sequence
  derived from LipC
GVAYIRWLLRARPAD

SEQ ID NO: 84 - protein, 15 aa, Artificial sequence
  derived from LipC
NDPQYQAELPEGSDT

SEQ ID NO: 85 - protein, 15 aa, Artificial sequence
  derived from LipC
RRVLYGDDPAQLLDV

SEQ ID NO: 86 - protein, 15 aa, Artificial sequence
  derived from LipC
VGIYGRYDWEDRSTP

SEQ ID NO: 87 - protein, 15 aa, Artificial sequence
  derived from LipC
RDASPIQRVTRNAPP

SEQ ID NO: 88 - protein, 15 aa, Artificial sequence
  derived from LipC
HRWPRHILDVKTAIA

SEQ ID NO: 89 - protein, 15 aa, Artificial sequence
  derived from LipC
AVSRSQVGYLELPGA

SEQ ID NO: 90 - protein, 15 aa, Artificial sequence
  derived from LipC
LLDGARTGPTAHAIA

SEQ ID NO: 91 - protein, 15 aa, Artificial sequence
  derived from Rv3452
PQFGSKTINLCNNGD

SEQ ID NO: 92 - protein, 15 aa, Artificial sequence
  derived from Rv3452
EVVFARGTGEPPGLG

SEQ ID NO: 93 - protein, 15 aa, Artificial sequence
  derived from Rv3452
PPASAGCPDAEVVFA

SEQ ID NO: 94 - protein, 15 aa, Artificial sequence
  derived from Rv3452
FVSSLRQQTNKSIGT

SEQ ID NO: 95 - protein, 15 aa, Artificial sequence
  derived from Rv3452
LPPAADDHIAAIALF

SEQ ID NO: 96 - protein, 15 aa, Artificial sequence
  derived from Rv3452
DGANDASDHIQQMAS

SEQ ID NO: 97 - protein, 15 aa, Artificial sequence
  derived from Rv1984
ASDDYRASASNGSDD

SEQ ID NO: 98 - protein, 15 aa, Artificial sequence
  derived from Rv1984
VSAPAGGRAAHADPC

SEQ ID NO: 99 - protein, 15 aa, Artificial sequence
  derived from Rv1984
GLGDVGEAFVDSLTS

SEQ ID NO: 100 - protein, 15 aa, Artificial sequence
  derived from Rv1984
SIGVYAVNYPASDDY

SEQ ID NO: 101 - protein, 15 aa, Artificial sequence
  derived from LipF
VRAAAAKNMVDGRPE

SEQ ID NO: 102 - protein, 15 aa, Artificial sequence
  derived from LipF
QRLQCDDEKPAAIVA

SEQ ID NO: 103 - protein, 15 aa, Artificial sequence
  derived from LipF
FIRDATADSSLSPVH

SEQ ID NO: 104 - protein, 15 aa, Artificial sequence
  derived from LipF
YQWLRARGYRPEQIV

SEQ ID NO: 105 - protein, 15 aa, Artificial sequence
  derived from LipF
DGRPEDLYEPLDHIE

SEQ ID NO: 106 - protein, 15 aa, Artificial sequence
  derived from LipF
LSPVHRSRYVAGSPR

SEQ ID NO: 107 - protein, 15 aa, Artificial sequence
  derived from LipF
ATRSLRQIGQFIRDA

SEQ ID NO: 108 - protein, 15 aa, Artificial sequence
  derived from LipF
SHSRIVNALSGFAES

SEQ ID NO: 109 - protein, 15 aa, Artificial sequence
  derived from LipF
PEQIVLAGDSAGGYL

SEQ ID NO: 110 - protein, 15 aa, Artificial sequence
  derived from LipF
LDHIESSLPPTLIHV

SEQ ID NO: 111 - protein, 17 aa, Artificial sequence
  derived from LipF
AGSPRAASRGAFGQSPI

SEQ ID NO: 112 - protein, 15 aa, Artificial sequence
  derived from LipF
GMALDDCHDAYQWLR

SEQ ID NO: 113 - protein, 15 aa, Artificial sequence
  derived from LipF
PPDSPRDVIVDNAVR

SEQ ID NO: 114 - protein, 15 aa, Artificial sequence
  derived from LipF
GDVKLYYEDMGDLDH

SEQ ID NO: 115 - protein, 15 aa, Artificial sequence
  derived from LipF
NRDVGLSTKTERHRP

SEQ ID NO: 116 - protein, 11 aa, Artificial sequence
  derived from LipF
GELTRNFSEAG

SEQ ID NO: 117 - protein, 15 aa, Artificial sequence
  derived from LipF
GSPAYPIPEDQVRAE

SEQ ID NO: 118 - protein, 15 aa, Artificial sequence
  derived from LipF
VDIRSGTAVSGDVKL

SEQ ID NO: 119 - protein, 15 aa, Artificial sequence
  derived from LipF
DNAVRVSKIIGSPAY

SEQ ID NO: 120 - protein, 15 aa, Artificial sequence
  derived from LipF
ERHRPGQPLATRLVR

SEQ ID NO: 121 - protein, 15 aa, Artificial sequence
  derived from LipF
LPRQLWDRVIGELTR

SEQ ID NO: 122 - protein, 15 aa, Artificial sequence
  derived from LipH
QLKTPPELLPELRIE

SEQ ID NO: 123 - protein, 15 aa, Artificial sequence
  derived from LipI
RLRDLPRQPVHPELR

SEQ ID NO: 124 - protein, 15 aa, Artificial sequence
  derived from LipI
IDDGIEAVRQRLRDL

SEQ ID NO: 125 - protein, 15 aa, Artificial sequence
  derived from Rv3451
RLQLHGGDGANDAIS

SEQ ID NO: 126 - protein, 15 aa, Artificial sequence
  derived from Rv3451
ADGCPDAEVTFARGT

SEQ ID NO: 127 - protein, 15 aa, Artificial sequence
  derived from Rv3451
TDPICHVGPGNEFSG

SEQ ID NO: 128 - protein, 15 aa, Artificial sequence
  derived from Rv3451
VNNRPIRLLTSGRAG

SEQ ID NO: 129 - protein, 15 aa, Artificial sequence
  derived from Rv3451
VFGNPSNRAGGSLSS

SEQ ID NO: 130 - protein, 12 aa, Artificial sequence
  derived from Rv3451
TAAPAPESLHGR

SEQ ID NO: 131 - protein, 15 aa, Artificial sequence
  derived from Rv3451
FVVQRLRAGSVPHLP

SEQ ID NO: 132 - protein, 15 aa, Artificial sequence
  derived from Rv3802c
HNPLTTDNQMSYNDS

SEQ ID NO: 133 - protein, 15 aa, Artificial sequence
  derived from Rv3802c
QGDLICAAPAQAFSP

SEQ ID NO: 134 - protein, 15 aa, Artificial sequence
  derived from Rv3802c
RQQGVGNQVPPSPRG

SEQ ID NO: 135 - protein, 15 aa, Artificial sequence
  derived from Rv3802c
HKPRPAFQDASCPDV

SEQ ID NO: 136 - protein, 15 aa, Artificial sequence
  derived from Rv3802c
AVVIMLRGAESPPSA

SEQ ID NO: 137 - protein, 15 aa, Artificial sequence
  derived from Rv3802c
AGDVASDIGNGRGPV

SEQ ID NO: 138 - protein, 15 aa, Artificial sequence
  derived from Rv3802c
MAKNSRRKRHRILAW

SEQ ID NO: 139 - protein, 15 aa, Artificial sequence
  derived from Rv3802c
LPPGPTPAHPHKPRP

SEQ ID NO: 140 - protein, 15 aa, Artificial sequence
  derived from LipM
SAGLWRRPAGGGTAK

SEQ ID NO: 141 - protein, 15 aa, Artificial sequence
  derived from LipM
RMLRIYRDYAHDGDI

SEQ ID NO: 142 - protein, 15 aa, Artificial sequence
  derived from LipM
ALTPNDPRFQPGFEE

SEQ ID NO: 143 - protein, 15 aa, Artificial sequence
  derived from LipM
SGLGPDRRTASAGLW

SEQ ID NO: 144 - protein, 15 aa, Artificial sequence
  derived from LipN
YLRDSDVDPADPRLS

SEQ ID NO: 145 - protein, 15 aa, Artificial sequence
  derived from LipN
KRDIDWFHTQYLRDS

SEQ ID NO: 146 - protein, 15 aa, Artificial sequence
  derived from LipN
DLSIPGPAGEIPARH

SEQ ID NO: 147 - protein, 15 aa, Artificial sequence
  derived from LipO
VCVSLNYRVSPRHTW

SEQ ID NO: 148 - protein, 15 aa, Artificial sequence
  derived from LipO
KRKFSTHRDIFVDAS

SEQ ID NO: 149 - protein, 15 aa, Artificial sequence
  derived from LipO
ANLADIWRRRDLPRD

SEQ ID NO: 150 - protein, 15 aa, Artificial sequence
  derived from LipO
ALRRGRRGDFGGLKG

SEQ ID NO: 151 - protein, 15 aa, Artificial sequence
  derived from LipO
MRFRRMARPRPLTRA

SEQ ID NO: 152 - protein, 15 aa, Artificial sequence
  derived from Rv2351c
SQLKLIRARFGVPVP

SEQ ID NO: 153 - protein, 15 aa, Artificial sequence
  derived from Rv2351c
MSRREFLTKLTGAGA

SEQ ID NO: 154 - protein, 15 aa, Artificial sequence
  derived from Rv2351c
TPPTAPPGTPGEFVT

SEQ ID NO: 155 - protein, 15 aa, Artificial sequence
  derived from Rv2351c
NNGLVQAFRQAADPR

SEQ ID NO: 156 - protein, 15 aa, Artificial sequence
  derived from Rv2351c
IMPENLEDAGVSWKV

SEQ ID NO: 157 - protein, 15 aa, Artificial sequence
  derived from LipQ
GPHRRYAAQTSDIPY

SEQ ID NO: 158 - protein, 15 aa, Artificial sequence
  derived from LipQ
PDFRDLVWHPTGEQS

SEQ ID NO: 159 - protein, 15 aa, Artificial sequence
  derived from LipQ
SANDPALQPGFESAD

SEQ ID NO: 160 - protein, 15 aa, Artificial sequence
  derived from LipQ
SDIPYGPGGRENLLD

SEQ ID NO: 161 - protein, 15 aa, Artificial sequence
  derived from LipQ
DLAPGRRAPVLIQVP

SEQ ID NO: 162 - protein, 15 aa, Artificial sequence
  derived from LipR
MNLRKNVIRSVLRGA

SEQ ID NO: 163 - protein, 15 aa, Artificial sequence
  derived from LipR
ICVDADKIETACAAS

SEQ ID NO: 164 - protein, 15 aa, Artificial sequence
  derived from LipR
RAPKGTRFQRVSIAG

SEQ ID NO: 165 - protein, 18 aa, Artificial sequence
  derived from LipR
RLRGHLHQSQGQPRGVVK

SEQ ID NO: 166 - protein, 15 aa, Artificial sequence
  derived from LipR
LDDGLDPKTTVIAGD

SEQ ID NO: 167 - protein, 15 aa, Artificial sequence
  derived from LipR
ACAASKTSIEHRRFA

SEQ ID NO: 168 - protein, 15 aa, Artificial sequence
  derived from Rv2350c
SWRIMPENLEDAGVS

SEQ ID NO: 169 - protein, 15 aa, Artificial sequence
  derived from Rv2350c
RGPLMVHDTFDHTST

SEQ ID NO: 170 - protein, 15 aa, Artificial sequence
  derived from Rv2350c
PFPQSMPTQETAPTR

SEQ ID NO: 171 - protein, 15 aa, Artificial sequence
  derived from Rv2350c
VVGYNGLVNDFKQAA

SEQ ID NO: 172 - protein, 15 aa, Artificial sequence
  derived from Rv2350c
GTPGEFVTVPDIDSV

SEQ ID NO: 173 - protein, 15 aa, Artificial sequence
  derived from Rv2350c
QENRSFDHYFGTLSD

SEQ ID NO: 174 - protein, 15 aa, Artificial sequence
  derived from Rv2350c
TDIEHIVLLMQENRS

SEQ ID NO: 175 - protein, 15 aa, Artificial sequence
  derived from Rv2350c
PNPSKPNLDHPRLNA

SEQ ID NO: 176 - protein, 15 aa, Artificial sequence
  derived from LipT
TAAADRRAALRVSNE

SEQ ID NO: 177 - protein, 15 aa, Artificial sequence
  derived from LipT
VALESATVGSMHERT

SEQ ID NO: 178 - protein, 15 aa, Artificial sequence
  derived from LipT
YLYRYDYAPRTLRWS

SEQ ID NO: 179 - protein, 15 aa, Artificial sequence
  derived from LipT
RIEFDPHQHRRIAWD

SEQ ID NO: 180 - protein, 15 aa, Artificial sequence
  derived from LipT
DDWPAYTQDDRAVLV

SEQ ID NO: 181 - protein, 17 aa, Artificial sequence
  derived from LipU
AIRSLRQIGEYIREATG

SEQ ID NO: 182 - protein, 15 aa, Artificial sequence
  derived from LipU
VASAAARNQVDGEPE

SEQ ID NO: 183 - protein, 15 aa, Artificial sequence
  derived from Rv2349c
RNGYVGSFKQAADPR

SEQ ID NO: 184 - protein, 15 aa, Artificial sequence
  derived from Rv2349c
GLMVHDRFDHTSQLQ

SEQ ID NO: 185 - protein, 15 aa, Artificial sequence
  derived from Rv2349c
TPTPLFQQKGWNPET

SEQ ID NO: 186 - protein, 15 aa, Artificial sequence
  derived from Rv2349c
GEWIPNSVDIDKVDG

SEQ ID NO: 187 - protein, 15 aa, Artificial sequence
  derived from Rv2349c
ISATVNPDGDQGGPQ

SEQ ID NO: 188 - protein, 15 aa, Artificial sequence
  derived from Rv2349c
PSPPNLDHPVRQLPK

SEQ ID NO: 189 - protein, 15 aa, Artificial sequence
  derived from Rv2349c
LGGLNDTSLSRNGYV

SEQ ID NO: 190 - protein, 15 aa, Artificial sequence
  derived from LipW
MSRTPPDIEVLTLES

SEQ ID NO: 191 - protein, 15 aa, Artificial sequence
  derived from LipW
PHYRLWNGRANRFGW

SEQ ID NO: 192 - protein, 15 aa, Artificial sequence
  derived from LipW
LTLESGVGVRLYRPA

SEQ ID NO: 193 - protein, 15 aa, Artificial sequence
  derived from LipW
DRLCLRFSSRLGITV

SEQ ID NO: 194 - protein, 15 aa, Artificial sequence
  derived from Rv1755c
NRGIPYRVPDPQIMP

SEQ ID NO: 195 - protein, 15 aa, Artificial sequence
  derived from Rv1755c
TPGEYVTVPDIDQVP

SEQ ID NO: 196 - protein, 15 aa, Artificial sequence
  derived from Rv1755c
GPQMVHDTFDHTSQL

SEQ ID NO: 197 - protein, 15 aa, Artificial sequence
  derived from Rv1755c
PQIMPTQETTPTRGI

SEQ ID NO: 198 - protein, 15 aa, Artificial sequence
  derived from Rv1755c
IDQVPGSGGIRGPIG

SEQ ID NO: 199 - protein, 15 aa, Artificial sequence
  derived from Rv2301
ALRSKVNKNVGVYAV

SEQ ID NO: 200 - protein, 15 aa, Artificial sequence
  derived from Rv2301
IELCHGDDPVCHPAD

(19) United States
(12) Patent Application Publication
(10) Pub. No.: US 2021/0199657 A1
(43) Pub. Date: Jul. 1, 2021

(54) IMMUNOGENIC PEPTIDES AND USE THEREOF

(71) Applicants: Omunis, Clapiers (FR); Centre Hospitalier Universitaire de Montpellier, Montpellier Cedex 5 (FR); AxLR, SATT du Languedoc Roussillon, Montpellier (FR); Institut National de la Sante et de la Recherche Medicale (Inserm), Paris (FR); Universite de Montpellier, Montpellier (FR)

(72) Inventors: Pierre-Alain Rubbo, Montpellier (FR); Edouard Tuaillon, Castelnau-le-lez (FR)

(21) Appl. No.: 16/079,629

(22) PCT Filed: Feb. 24, 2017
(86) PCT No.: PCT/FR2017/050418

§ 371 Date: Aug. 24, 2018

(30) Foreign Application Priority Data
Feb. 26, 2016 (FR) 1651610

(51) Int. Cl. G01N 33/569; C07K 14/35

(52) U.S. Cl. CPC G01N 33/5695; G01N 2800/26; G01N 2333/35; C07K 14/35

(57) ABSTRACT

The invention relates to an in vitro method of screening immunogenic peptides of interest, capable of recognizing antibodies originating from the serum of individuals suffering from active tuberculosis, said method comprising:—bringing into contact of peptides of the serum originating from patients suffering from active tuberculosis—detecting the formation of immune complexes in the preceding step, and—selecting peptides of interest for which the value of a ratio R is greater than or equal to 1.5, the ratio R being the measurement value of the formation of immune complexes to the measurement value obtained from the control sample.

The present invention relates to immunogenic peptides and the use thereof, in particular in the context of diagnosing pathologies.

With one third of the world's population infected and one death every 20 seconds, tuberculosis (TB) remains one of the deadliest diseases in the world. There is currently no rapid, precise diagnostic test that has been validated according to the expectations of the health authorities, and in particular the World Health Organization (WHO), the reference test requiring several weeks to yield a result.

Today, the international reference methods for diagnosing TB are long (up to 8 weeks for bacteriological culture), low-performing, expensive and difficult to implement, since they use lung samples, and require a laboratory with security level 3 and hospitalization of the patient.

For the first time in its history, in 2011 the WHO published an explicitly negative general policy recommendation regarding the use of the first generation of serological tests marketed for the diagnosis of TB (WHO 2011, commercial serodiagnostic tests for diagnosis of TB: policy statement).

More than one million of these tests, not clinically validated and with poor performance, were sold each year worldwide, in particular in countries in the southern hemisphere (Africa, Asia), taking advantage of weak local regulatory constraints. Nearly all of these tests use the same antigens, and they were never subject to rigorous clinical validation, leading to very weak performance with a risk for patients.

The WHO is thus opening the door for an opportunity for a new generation of tests satisfying strict quality and clinical validation criteria.

During the latent infection, the TB bacillus is contained in a macrophage qualified as foamy due to the microscopic appearance of its cytoplasm saturated with lipid vacuoles. Mycobacterium tuberculosis stores fatty acids in the form of triglycerides (TG) allowing bacteria to enter the dormant phase (latent TB). These lipid vacuoles make up a source of carbon for bacteria and are used by the latter to leave dormancy and be reactivated (active TB). The reduction in TG levels during the reactivation of TB coincides with the increase in the activity of certain proteins breaking down the TGs. Some of these proteins, studied relatively little thus far, appear to be closely linked to the reactivation of TB from latent forms and are therefore of major interest in the early identification of active TB or to monitor latent TB cases with a high risk of developing the serious form of the disease.

Patent application WO/2012/164088 is known from the state of the art and teaches a method for diagnosing active tuberculosis based on ELISpot B, using lipase proteins with lipolytic activity.

However, although such a method is effective in the early detection of active tuberculosis, it can only be implemented with heavy equipment in an equipped laboratory and requires specific technical skills, and therefore cannot be used easily in the field with at-risk populations.

There is therefore a need to provide a simpler method that retains a level of active TB detection sensitivity at least as good as that obtained with the method of application WO/2012/164088.

One aim of the invention is therefore to provide a method for diagnosing active tuberculosis that can be implemented easily in all situations, and in particular without highly equipped hospital infrastructure.

Another aim of the invention is to determine the best peptide candidates making it possible to carry out this new fast and effective diagnostic method.

Therefore, the invention relates to an in vitro method of screening at least one immunogenic peptide of interest capable of recognizing at least one antibody originating from the serum of individuals suffering from active tuberculosis, said at least one immunogenic peptide being a hydrophilic peptide originating from a hydrophobic protein, said hydrophobic protein being a wall protein, or secreted from bacteria from the Mycobacterium genus, said hydrophobic protein having a lipolytic activity, said method comprising the following steps:
bringing into contact of at least one hydrophilic peptide originating from at east one hydrophobic peptide with
   successively at least two independent pools of serums originating from patients suffering from confirmed active tuberculosis, to allow the formation of immune complexes between said antibodies and said peptides to be screened, and
   at least one control sample originating from an individual not suffering from tuberculosis,
detecting the formation of immune complexes in the previous step,
conducting a first selection of the peptides of interest for which the value of a ratio R is greater than or equal to 1.5 for at least one of the pools of independent serums originating from patients suffering from confirmed active tuberculosis,
   the ratio R being the normalized measurement value of the formation of immune complexes, relative to the respective background noises of the serums and components used to detect the immune complexes, to, or divided by, the normalized measurement value obtained from the sample originating from a healthy individual relative to the respective background noises of the serums and components used to detect the immune complexes.

The invention is based on the surprising observation made by the inventors that certain peptides originating from determined proteins are very good candidates for carrying out a method for diagnosing active tuberculosis, while proposing a sensitivity and an efficacy for detecting the pathology at a high level.

The diagnosis proposed by the invention can be done in humans as well as animals.

Hereinafter, tuberculosis will be referred to uniformly as “tuberculosis” or “TB”.

In the invention, the terms “hydrophobic protein having a lipolytic activity” are used to refer to an enzyme with lipolytic activity and include phospholipases A, B, C or D, and lipases, in particular triglyceride lipases, lipases or diglycerides, monoglyceride lipases.

During the infection, Mycobacterium tuberculosis accumulates intracellular inclusion bodies charged with lipids, the lipids of which probably originate from the degradation of the cell membrane of the host. One then has strong evidence supporting the fact that fatty acids are a source of carbon during dormancy. Mycobacterium tuberculosis stores fatty acids in the form of triacylglycerol (TAG) when it enters the nonreplicating persistence stage (latent stage). Furthermore, granulomas containing foamy macrophages, which are cells containing, in their cytoplasm, a large quantity of neutral lipids surrounded by phospholipids, have been found, These lipid bodies are induced by the internalization of the bacteria, and therefore provide a source of carbon for the survival and reactivation of the pathogen. More generally, these discoveries support the fact that the enzymes involved in the degradation of the lipids can assume significant physiological functions and may participate in the extraordinary survival capability and the reactivation of Mycobacterium tuberculosis from infected cells. The degradation of the lipids of the host by Mycobacterium tuberculosis is probably done by lipolytic enzymes, such as lipases and phospholipases, including the family of cutinase enzymes.

Lipases are water-soluble proteins having a lipolytic activity belonging to the group of esterases and catalyzing the hydrolysis of substrates insoluble in water, like the ester bonds of triacylglycerol and phospholipids.

In this context, the lipolysis catalytic reaction involves different processes at the interface and closely depends on the structure of the lipid substrates present in oil-in-water emulsions, membrane bilayers, micelles and vesicles. The catalytic process can be described as a reversible step of adsorption/desorption of the lipases taking place in the oil/water interface, followed by the formation of an enzyme/substrate complex at the interface and the release of the lipolysis products. Among the identified M. tuberculosis lipases, 24 have been classified in the family of enzymes called “Lip family”. However, this classification is based solely on the presence of the GXSXG consensus sequence, which is characteristic of esterases and members of the hydrolase family having α/β folds.

In the invention, the method for detecting immunogenic peptides is therefore based on taking advantage of the properties of proteins with a lipolytic activity (which make it possible to detect active tuberculosis) while doing away with the difficulty of working with whole proteins, which can be difficult to manipulate and/or expensive and complex to produce.

Indeed, it is particularly relevant to find small immunogenic fragments of said proteins with lipolytic activity, since they are membrane proteins (inserted into the lipid bilayer) and are therefore hydrophobic, or simply because they are proteins degrading the lipids, therefore hydrophobic.

In the invention, “hydrophobic protein” refers to a protein that is considered, as a whole, to have little affinity for aqueous solutions and that would have little ability to dissolve in an aqueous liquid.

Proteins are made up of amino acids that may be polar (hydrophilic) or hydrophobic. When the protein is synthesized, it adopts a specific three-dimensional configuration related to its activity or its function such that the amino acids far away from one another in the sequence can be found close to one another in space. If, during the folding of a protein, all of the polar amino acids are found within a pocket that is surrounded by hydrophobic amino acids, the whole protein will then be considered hydrophobic, even if the proportion of hydrophilic amino acids is higher than that of hydrophobic amino acids.

Likewise, a protein will be considered hydrophilic if its three-dimensional configuration is such that the majority of the amino acids present on the surface of the protein are hydrophilic.

The notion of hydrophily and hydrophobicity of a protein are well known by those skilled in the art. It is also possible for one skilled in the art, from the primary sequence of a protein, to determine its hydrophily/hydrophobicity profile by predicting its structure and its hydrophobicity index.

In the invention, “hydrophilic peptide originating from a hydrophobic protein” refers to a fragment of said hydrophobic protein, as defined above, whose properties are to be easily soluble and stable in an aqueous liquid, i.e., in water or polar solvents.

In order to predict the hydrophilic peptides to be screened according to the invention from hydrophobic proteins, it is in particular possible to base oneself on the solubility of said peptides. Sequences having many basic residues without intercalated acid residues (acid/basic balance) may be difficult to solubilize. As a result, a balance is determined in order to analyze the solubility of each of the peptide sequences, which may be immunogenic after screening.

It is therefore important to choose the peptides whose acid/base balance B_{ab} is the greatest, using the formula:

B_{ab}=Aₐₐ−B_{bb}

where
Aₐₐ=(Nₐ/N)×100 with Nₐ=the number of acid residues of amino acids in the sequence and N=the number of amino acids, and
B_{bb}=(N_{b}/N)×100 with Nₐ=the number of basic amino acid residues in the sequence and N=the number of amino acids.

The solubility can also be predicted by counting the number of charged residues and adding the free terminal ends of the peptide thereto. Theoretically, at least one charge every 5 residues is required to obtain a minimal solubility. It is also necessary to avoid linking more than 3 to 4 hydrophobic residues. The hydrophobicity at pH 6.8 makes it possible to verify solubility of the peptide in an aqueous buffer. This value makes it possible to verify the compatibility with the coupling buffers during the step for conjugation to the carrier protein.

It will be noted that the hydrophobicity at pH=6.8 corresponds to the value of the hydrophobicity of each of the amino acids at pH=6.8 to the total number of amino acids of the considered peptide.

It may in particular be advantageous to verify, before testing their immunogenicity, that the peptides to be screened are for example i) flexible, i.e., not spatially constrained, i.e., with epitopes that are freely accessible for any antibodies relative to the general structure of the peptide, ii) if they are located in retained protein patterns or secondary structures (helixes, 3 folds), which would decrease their specificity, iii) if they are found in exposed regions on the whole protein from which they are derived. One skilled in the art may also find other appropriate characteristics that could complete his choice of potentially immunogenic peptides.

Once the hydrophilic peptides are identified, their immunogenicity is then tested using the following method:
1—each peptide is placed in contact with at least two pools of serums, the serums originating from patients suffering from clinically confirmed active TB, and
2—in parallel with the control sample originating from a healthy individual, who is not suffering from tuberculosis, in particular active tuberculosis, i.e., an individual who has never been in contact with tuberculosis or a patient who is in the latent phase of tuberculosis (and has therefore not developed active tuberculosis).

The objective is to determine whether the tested peptides are capable of forming immune complexes with at least one antibody contained in said pools of serums from individuals suffering from active TB, which means that the peptides are potentially mutagenic.

The potential immune complexes are detected according to traditional methods that consist of marking and identifying the presence of an immune complex detecting the constant part of the antibodies that have potentially interacted with the peptides to be screened using specific immunoglobulins of the constant parts of the antibodies coupled with markers allowing a quantification.

A traditional laboratory method for detecting these immune complexes consists of an ELISA (Enzyme-Linked ImmunoSorbent Assay) test. This detection method can also be adapted on different solid substrates in order to facilitate the identification of immune complexes outside of laboratories.

In order to determine which peptides are of interest, a ratio R is calculated, the ratio R being the normalized measurement value of the immune complex formation, relative to the respective background noises of the serums and components used to detect the immune complexes, to, or divided by, the normalized measurement value obtained from the sample derived from a healthy individual relative to the respective background noises of the serums and components used to detect the immune complexes.

This means that the following formula is applied:

$$R = \frac{( Vp + e - VBlanc ) - ( Vs + e - VBlanc )}{( Vp + n - VBlanc ) - ( Vs + n - VBlanc )}$$

where:
Vp+e corresponds to the value measured during the detection of the human complex when the peptide (p) is placed in contact with the serum of a patient suffering from active TB (e),
Vs+e corresponds to the value measured upon the detection of a human complex when the solvent of the peptide (s) is placed in contact with the serum of a patient suffering from active TB (e),
Vp+n corresponds to the value measured upon the detection of an immune complex when the peptide (p) is placed in contact with the serum of a healthy individual (n),
Vs corresponds to the value measured upon the detection of an immune complex the solvent of the peptide (s) is placed in contact with the serum of a healthy individual (n), and
VBlanc corresponds to the value measured upon the detection of an immune complex in the absence of any serum whatsoever, peptide and solvent.

The values are said to be normalized, since for each measurement, account is taken of the potential background noise of each biological material: the serum (the positive serums are compared with the serum of a healthy individual), the peptide (the peptide is compared with its solvent), etc.

Irrespective of the method used to obtain the ratio R, if, for a determined peptide, the ratio as calculated above is greater than or equal to 1.5, the peptide will be considered particularly interesting and capable of effectively detecting marker antibodies for active TB. On the contrary, if the ratio R is less than 1.5, it will not be used.

In the invention, as mentioned above, each peptide is placed in contact with at least two pools of serums. In a first approach, a pool or mixture of several serums is used originating from separate patients each with clinically proven active TB (positive microbiological culture results—reference clinical test). These pools make it possible to have a mixture of serums and thus to increase the diversity of antibodies that can be detected.

Independent pools are used, i.e,, mixtures of serums that do not have the same origins. For example, if a first pool includes 4 serums coming from 4 different individuals, a second independent pool will comprise several serums, none of which will be in common with at least one of the serums from the first pool.

Advantageously, as mentioned above, the immune complexes between the peptide and the antibodies contained in the pools are quantified by immunodetection by using immunoglobulins coupled with a detection agent. It is for example possible, depending on the marker used, to measure the optical density OD. Depending on the marker used, one skilled in the art will know how to determine the best method for quantifying the immune complexes.

In the invention, the preferred peptides are those which have a ratio R greater than 1.5 for all of the pools of the at least two pools of serums. Of course, the peptides that have a ratio R greater than or equal to 1.5 for a pool of the at least two pools of serums will also be of interest. Conversely, the peptides for which the ratios R, irrespective of the considered pool of the at least two pools of serum, are below 1.5, will not be selected.

In one advantageous embodiment, the invention relates to the aforementioned screening method, further including the following steps:
bringing the peptides selected in the first selection step into contact with each of the individual serums making up said independent pools of serums originating from patients suffering from confirmed active tuberculosis, to allow the formation of immune complexes between said antibodies and said peptides to be screened,
detecting the formation of immune complexes in the preceding step, and
carrying out a second selection of peptides of interest for which the value of the ratio R is greater than or equal to 1.5 for with each of the individual serums making up said independent pools of serums for patients with confirmed active tuberculosis.

Advantageously, once a peptide has been identified using the aforementioned method, it is also advantageous to conduct a second reactivity test with, individually, each of the serums making up the at least two pools. Such double screening confirms the selection, and makes it possible to potentially eliminate selected peptides that would only recognize antibodies that are overrepresented in one of the serums of the pools.

Like during the first screening, the ratio R is measured according to the aforementioned formula, and the peptides for which the ratio R is greater than or equal to 1.5 are selected as being the best performing peptides, i.e., the peptides that have a strong affinity for specific antibodies for active tuberculosis (active TB).

Advantageously, the invention relates to the aforementioned method in which, during the first selection, only the peptides for which the value of a ratio R is greater than or equal to 1.5 for at least two of the independent pools of serums coming from patient suffering from confirmed active tuberculosis are selected.

It is of course particularly interesting and advantageous to select the peptides for which, during the first screening in a pool, the ratio R is greater than or equal to 1.5, for each of said at least two pools, and for which the ratio R for each of said serums making up said at least two pools is greater than or equal to 1.5.

In another advantageous embodiment, the invention relates to the method as defined above, in which the hydrophilic peptides are identified by bioinformatics, based on their apparent hydrophily.

As discussed above, different criteria can be used to determine the potential hydrophilic peptides that must be tested to determine their immunogenicity according to the inventive method. This peptide selection can be done by informatics by estimating different relevant criteria for one skilled in the art, such as the hydropathy, stability, solubility, secondary structure, accessibility of the peptide in the whole protein, flexibility, etc. One skilled in the art will know how to choose the most relevant criterion or criteria to determine whether the peptide is considered hydrophilic within the meaning of the invention and whether it should be screened using the aforementioned method.

According to another advantageous embodiment, the invention relates to the aforementioned method, in which the hydrophilic peptides have a size from 15 to 2.5 amino acids.

The peptides to be screened in the invention are peptides with an average size from 15 to 25 amino acids, i.e., having 15, 16, 17, 18, 19, 20, 21, 22, 23, 24 or 25 natural amino acids.

These peptides may also be modified on one or several amino acid residues. For example, by protecting certain residues such as the cysteine II residues it is possible to add, on these peptides, other N-terminal or C-terminal chemical groups making them easier to manipulate and use in a rapid test (grafting to the substrate, epitopic presentation, conformation, etc.). These groups may for example be Biotin, soluble “carrier” protein of the BSA, thiol or NH2 type as long as it is not present in the peptide sequence of interest.

Advantageously, the invention relates to the method previously described in which the immune complexes are detected by immunodetection using marked antibodies against the constant part of the immunoglobulins.

The invention also relates to at least one hydrophilic peptide, intended to detect active tuberculosis in a patient's blood sample, capable of being obtained or screened using the method described above.

Furthermore, the invention relates to a peptide capable of being screened using the aforementioned method, for use in the context of diagnosing active tuberculosis in an individual.

The invention further relates to at least one hydrophilic peptide, comprising from 15 to 25 amino acids, originating from a hydrophobic protein, said hydrophobic protein being a bacterial wall protein from the Mycobacterium genus, said hydrophobic protein having a lipolytic activity.

The invention also relates to a peptide a hydrophilic peptide, including from 15 to 25 amino acids, originating from a hydrophobic protein, said hydrophobic protein being a bacterial wall protein from the Mycobacterium genus, said hydrophobic protein having a lipolytic activity, for use thereof in the context of diagnosing active tuberculosis in an individual.

As previously mentioned, the peptides screened using the aforementioned method are particularly advantageous to carry out a method for diagnosing active tuberculosis in individuals. Indeed, since these peptides are selected for their ability to detect antibodies specifically present in the serum of patients suffering from active tuberculosis, they will be particularly effective in determining the serological status of an individual with respect to tuberculosis.

In one advantageous embodiment, the invention relates to a hydrophilic peptide, as defined above, said peptide being represented by any one of the following sequences: SEQ ID NO: 1. to SEQ ID NO: 30.

In the invention, SEQ ID NO: 1 to SEQ ID NO: 30 refers to the following sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.

The most advantageous peptides of the invention are the peptides with sequence: SEQ ID NO: 1, SEQ ID NO: 2, SEQ ID NO: 3, SEQ ID NO: 4 and SEQ ID NO: 5, which have ratios R significantly greater than 1.5 for four independent pools of serums.

Peptides SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19 are also interesting inasmuch as the ratios R are greater than 1.5 for 3 of the 4 serums tested.

The invention also advantageously relates to a composition comprising at least one of the peptides chosen from among the peptides with the following sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30, for use thereof in the context of diagnosing active tuberculosis in an individual.

Advantageously, the invention relates to a composition for the aforementioned use, including at least one of the peptides chosen from among the peptides with the following sequences: SEQ ID NO: 1, SEQ ID NO: 2, SEQ ID NO: 3, SEQ ID NO: 4 and SEQ ID NO: 5.

Advantageously, the invention relates to a composition for the aforementioned use, including at least one of the peptides chosen from among the peptides with the following sequences: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19.

The invention also relates to an in vitro method of diagnosing an individual who may be suffering from active tuberculosis, said method including:
a step of bringing a blood sample from said individual into contact with at least one hydrophilic peptide as defined above, and
a step of detecting an immune complex between at least one antibody of said blood sample and said peptides.

According to another aspect, the invention relates to a method for diagnosing active tuberculosis in an individual including a step for bringing a blood sample, in particular serum, from said individual into contact with at least one peptide chosen from among the peptides with sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30, and

a step for detecting at least one immune complex between at least one antibody of said blood sample and said at least one peptide.

The invention further relates to a kit for diagnosing active tuberculosis, including:
at least one hydrophilic peptide as defined above, and
means for identifying immune complexes between at least one antibody originating from a blood sample of an individual and said at least one peptide.

In the kit according to the invention, said at least one peptide is in particular a peptide chosen from among the peptides with sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.

Means for detecting immune complexes may involve providing immunoglobulins specifically recognizing the constant part of the antibodies, in particular human antibodies, said immunoglobulins in particular being able to be coupled with fluorochromes, enzymes, etc. One skilled in the art knows what types of marked immunoglobulins are appropriate for producing such a kit and performing the detection of immune complexes.

Advantageously, the invention relates to a kit as defined above, comprising means for identifying immune complexes between at least one antibody originating from a blood sample of an individual are arranged on a chromatographic-type substrate. Other formats, such as magnetic balls or “electrosensors”, can also be used.

According to one advantageous aspect of the invention, a kit is provided for detecting active tuberculosis in the form of a portable kit, usable in all situations and in all situations, by depositing a drop of blood. Such a kit is a rapid detection kit, in just a few minutes. An example of such a portable kit is illustrated in FIG. 1.

Advantageously, the invention relates to the aforementioned kit, further including at least one positive control. Whether it involves a kit to be used in a laboratory, or above all a portable kit, it is particularly relevant to have a positive control, i.e., a serum originating from one or several patients whose active tuberculosis is or has been clinically confirmed.

The possibility of diagnosing active TB simply, reliably and quickly from venous or capillary blood is a medical and economic success at least identical to that of rapid tests for detecting other infections (for example, HIV). Although the specificity of the test is not optimal, a good predictive negative value allows a test of this type very widespread first-line use for screening the disease and providing orientation toward a confirmation test. The development of a rapid test for screening or diagnosis of active TB contributes quite significantly to meeting objectives set by the WHO for the eradication of tuberculosis.

In another advantageous embodiment, the invention relates to the aforementioned kit, wherein said at least one hydrophilic peptide is coupled with magnetic nanoshells.

The coupling of the peptides according to the invention with magnetic nanoshells makes it possible to obtain results similar to a traditional ELISA immunological test, with a smaller quantity of peptides and above all in less than 10 minutes (versus approximately 2 hours for a traditional ELISA). The advantage of such coupling is that it limits the wash times necessary to eliminate the nonspecific interactions, since the immune complexes are isolated by magnetisms owing to the shells on which they are formed.

The invention further relates to a hydrophilic peptide as defined above, in particular a peptide essentially consisting of or consisting of any one of sequences SEQ ID NO: 1 to 30, for use thereof in the context of diagnosing active tuberculosis in an individual.

As mentioned above, the specific peptides of the invention are very appropriate for detecting antibodies against Mycobacterium tuberculosis peptides, and therefore for making it possible to make an active tuberculosis diagnosis in an individual.

The invention will be better understood in light of the following figures and examples:

### LEGEND OF THE FIGURES

FIG. 1 shows an example portable kit according to the invention.

FIG. 2 shows a diagram representative of the embodiment of FIG. 1.

FIG. 3 is a schematic depiction of the visual result obtained upon a positive diagnosis of active tuberculosis (++) or upon a negative diagnosis (−). T designates the significant marking of positivity of the sample, and T+ indicates the positive control of the reaction. The arrow indicates the migration direction.

FIGS. 4 to 7 show histograms of the reactivity of the tested peptides in index form (ratio R according to the invention).

FIG. 8 shows a histogram of the reactivity of peptides C2, C12, M3, V5 and G9 (respectively shown by sequences SEQ ID NO: :1 to 5) in index form (ratio R according to the invention). The bars show the 4 pools tested for each of the peptides.

FIG. 9 depicts a histogram showing the comparison of the results obtained between the traditional ELISA technique (columns in dark gray) and the nanoshell technology (column in light gray), using the peptide SEQ ID NO: 3. Samples 1 and 2 correspond to positive controls, samples 3 and 4 correspond to negative samples, samples 5 to 7 correspond to sera from patients and sample 8 corresponds to a sample announced as negative.

FIG. 10 depicts a histogram showing the comparison of the evaluation results of 19 human sera (13 positives with active TB, samples 1 to 27, and 6 noninfected negatives, samples Neg1 to Neg6) between the “lab table” version (black columns) from the laboratory and the “transportable” version of the prototype developed (gray columns).

FIG. 11 shows a histogram showing the average of the signals obtained for the analysis of 20 samples tested in single blind (15 positives and 5 negatives) on the optimized transportable prototype. The positive samples are nos. 1, 3, 4, 5, 7, 8, 10, 11, 12, 14, 15, 16, 17, 19, 20. The negative samples are nos. 2, 6, 9, 13, 18.

FIG. 12 depicts a histogram showing the evaluation of the stability of the magnetic nanoshells diagnostic candidate peptides coupling over time, on negative (Neg6) or positive (Pos1) samples. The histogram here shows the evaluation of two patient serum samples (one negative and one positive) with the same technique on magnetic nanoshells using the same peptide (M3) grafted at different time intervals on the nanoshells. Both will have been analyzed with the grafted peptide and tested on Nov. 24, 2016 (right histogram for each of the two patients) compared with the same peptide grafted on Sep. 12, 2017 and tested on Nov. 30, 2016 and Dec. 1, 2016 (first two histograms for each of the two patients). A decrease appears in the signal over time for this peptide, which may be explained by an instability of the peptides in solution or a lack of reproducibility of the grafting.

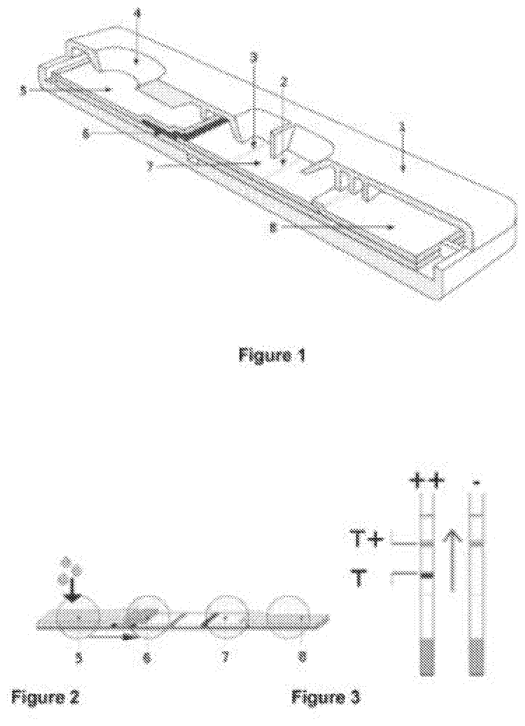

If reference is made to FIG. 1, the kit for diagnosing active tuberculosis according to the invention is made up of a device comprising a cassette 1, including three windows 2; 3; 4 respectively making it possible to detect the reactivity of a sample with positive control, to detect the reactivity of a sample with at least one peptide according to the invention, and to deposit a sample of serum to be tested. The cassette 1 covers a reservoir including, positioned below the window 4,
a sample substrate 5 allowing the macromolecular filtration of the deposited sample (blood, serum, human plasma) and making it possible to monitor the matrix (ionic force, absorption speed, etc.),
a conjugation substrate 6 including detection antibodies against the antibodies that may be contained in the sample to be tested, and coupled with a tracer such as colloidal gold, latex, carbon.

Positioned below the windows 3 and 2 is a membrane 7 made from nitrocellulose, nylon or polyvinylidene fluoride (PVDF) on which is fixed, in the form of a line, at least one peptide according to the invention (below the window 3) and a line of biotinylated bovine albumin (below the window 2).

The reservoir further includes, juxtaposed with the membrane 7, an absorbent substrate 8 whose function is to serve as a residual liquid reservoir and for stabilization of the migration speed.

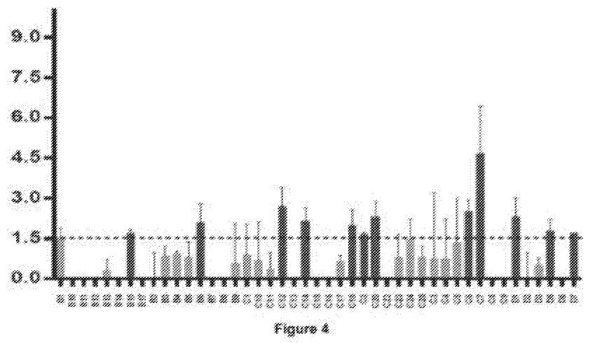

FIG. 2 shows the device of FIG. 1 in use. First, a biological sample (in particular blood or serum) is deposited on the sample substrate 5 via the window 4. Under the effect of the capillarity generated by an absorbent substrate 8 and a specific migration buffer facilitating the capillarity, the content of the sample is gradually, at a constant speed, transferred to the conjugation substrate 6, where the antibodies of the biological sample are then capable of coupling with the detection antibody.

Still under the effect of the capillarity, the content of the sample migrates from the conjugation substrate 6 to the membrane 7. When they pass over the peptide line according to the invention, the antibodies against the peptide of the invention are immobilized by the immunological reaction, the rest of the sample continuing to migrate. The same is true for the control line.

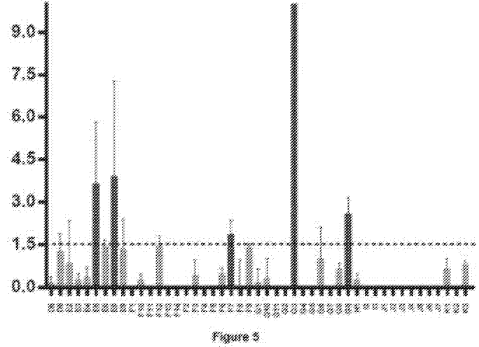

As indicated in FIG. 3, when the sample to be tested comprises antibodies against the peptides of the invention that have been deposited on the membrane 7, the latter are immobilized and accumulate, and owing to the coupling with the marked antibodies, it is possible to see an indicator line of the reactivity appear, at the test zone T.

### EXAMPLES

### Example 1—Identification of the Peptides to be Screened

From the 25 recombinant proteins in the family of lipolytic enzymes, the inventors have performed epitope screening in order to hierarchize, in. silico, more than 800 peptides based on different characteristics: hydrophobicity, secondary structure, etc. This ranking has allowed them to select the 200 candidates that gather the most promising characteristics for the diagnosis of active tuberculosis. The immunogenicity of each of the 200 peptides was next evaluated in an ELISA test for the screening of these peptides. From the results obtained on patient pools, the 30 best candidates were retained for the continuation of technical evaluations.

Using a computer algorithm and from provided genetic sequences, the main characteristics of the diagnostic candidate proteins were identified and a classification of the best potential peptides was established. In all, the inventors obtained a list of 833 overlapping peptides of 15 amino acids classified in 4 groups (1, 2, 3 and “bad”) based on their in siiico theoretical immunogenicity (with 1 being the best and “bad” being the worst). The information regarding the 200 tested peptides is indicated in the following table 1:

TABLE 1
List of 200 tested peptides

| | | | | | SEQ |
|---|---|---|---|---|---|
| | | | | Peptide | ID |
| | Proteins | Peptides | AA position | sequences | NO: |
| 1 | LipC (Rv0220) | C2 | 71-85aa | DLLTPGINEVRRRDR | 1 |
| | LipC (Rv0220) | C12 | 321-335aa | RNAPPFLVIHGSRDC | 2 |
| | LipM (Rv2284) | M3 | 131-145aa | GGTAKTPGPLRMLRI | 3 |
| | Rv2349c(PLCC) | V5 | 351-365aa | TPLTAPEGTPGEWIP | 4 |
| | LipG (Rv0646c) | G9 | 141-155aa | KTLAVIFSSNNHRFL | 5 |
| 2 | LipO (Rv1426c) | 7 | 301-315aa | FTTDAPGRREFVGLL | 6 |
| | Rv2301 | Z1 | 31-45aa | TAACPDAEWFARGR | 7 |
| | Rv1984 | E8 | 101-115aa | QRTVASCPNTRIVLG | 8 |
| | Rv3452 | D7 | 191-205aa | CNNGDPICSDGNRWR | 9 |
| | Rv3452 | D1 | 1-15aa | MIPRPQPHSGRWRAG | 10 |
| | Rv2351c (PLCA) | P2 | 91-105aa | AGVTIPFRLDTTRGP | 11 |
| | LipT (Rv2045c) | T9 | 481-495aa | RAVLVFDRRCRIEFD | 12 |
| | LipW (Rv0217c) | W3 | 81-95aa | GYVMGTAQQDDRLCL | 13 |
| | LipW (Rv0217c) | W5 | 181-195aa | DDRPSIAPANPHYRL | 14 |
| | LipW (Rv0217c) | W7 | 211-225aa | DADARVAVPRRDDL | 15 |
| | Rv0183 | B15 | 251-265aa | MPRRAPALTAPLLVL | 16 |
| | LipC (Rv0220) | C14 | 61-75aa | ASDFLSATAKDLLTP | 17 |
| | LipC (Rv0220) | C18 | 221-235aa | VDKFGGDRNFIAVAG | 18 |
| | Rv1984 | E5 | 31-45aa | HADPCSDIAVVFARG | 19 |
| | LipC (Rv0220) | C6 | 141-155aa | QLLDVWRRKDMPTKP | 20 |
| | LiPc (Rv0220) | C7 | 241-255aa | HLSALAGLTANDPQY | 21 |
| | Rv3452 | D5 | 161-175aa | AIALFGNPSGRAGGL | 22 |
| | Rv2301 | Z3 | 161-175aa | NFSPAYNDRTIELCH | 23 |
| | LipF (Rv3487c) | F7 | 1-15aa | VRAPGVRAADGAGRV | 24 |
| | Rv0183 | B6 | 271-285aa | RLIPIEGSRRLVECV | 25 |
| | LipG (Rv0646c) | G3 | 51-65aa | AKGLRVIRYDNRDVG | 26 |
| | Rv2351c (PLCA) | P6 | 341-355aa | DENGGFFDHVTPPTA | 27 |
| | Rv2350c(PLCB) | S13 | 501-512aa | TAPTRGIPSGLC | 28 |
| | LipT (Rv2045c) | T6 | 451-465aa | RVSNEVQRRWRCFSQ | 29 |
| | LipC (Rv0220) | C20 | 281-295aa | DRSTPERARFVDFLE | 30 |
| 3 | Rv0183 | B1 | 1-15aa | MWAEKSPRRSSAGSR | 31 |
| | Rv0183 | B16 | 281-295aa | LVECVGSADVQLKEY | 32 |
| | LipC (Rv0220) | C1 | 1-15aa | MNQRRAAGSTGVAYI | 33 |
| | LipC (Rv0220) | C10 | 301-315aa | RTIDRHPEVFRDASP | 34 |
| | LipC (Rv0220) | C4 | 111-125aa | APERTPPVCGALRHR | 35 |
| | LipC (Rv0220) | C24 | 361-375aa | ELPGAGHGFDLLDGA | 36 |
| | LipC (Rv0220) | C3 | 101-115aa | SPDDLAVEWPAPERT | 37 |
| | LipC (Rv0220) | C5 | 121-135aa | ALRHRRYVHRRRVLY | 38 |
| | LipC (Rv0220) | C21 | 331-345aa | GSRDCVIPVEQARSF | 39 |
| | LipC (Rv0220) | C19 | 261-275aa | EGSDTSVDAVVGIYG | 40 |
| | Rv3452 | D9 | 91-105aa | PANGDFLAAADGAND | 41 |
| | Rv1984 | E2 | 91-105aa | NGSDDASAHIQRTVA | 42 |
| | Rv1984 | E9 | 201-217aa | MTSQAATFAANRLDHAG | 43 |
| | Rv1984 | E1 | 41-55a | VFARGTHQASGLGDV | 44 |
| | LipF (Rv3487c) | F12 | 131-145aa | PNiGTDAMFPARAFD | 45 |
| | LipI (Rv1400c) | I1 | 1-15aa | MPSLDNTADEKPAID | 46 |
| | Rv3802c | K5 | 71-85aa | SCPDVQMISVPGTWE | 47 |
| | LipO (Rv1426c) | O3 | 121-135aa | ATLPTEPMRSRGRNL | 48 |
| | LipO (Rv1426c) | O6 | 271-285aa | TPNDPRFQPGFEQVD | 49 |
| | Rv2351c(PLCA) | P9 | 501-512aa | TPVRGTPSGLCS | 50 |
| | Rv2351c(PLCA) | P3 | 101115aa | TTRGPFLDGECVNDP | 51 |
| | LipQ (Rv2485c) | Q6 | 201-215aa | ICVSINYSKSPRCTW | 52 |
| | LipQ (Rv2485c) | Q9 | 341-355aa | GEKDPMVPSAQSRAF | 53 |
| | LipQ (Rv2485c) | Q10 | 401-415aa | IYGRRMGARKGSLAL | 54 |
| | LipQ (Rv2485c) | Q4 | 151-165aa | ENLLDIWRRPDLAPG | 55 |
| | LipQ (Rv2485c) | Q8 | 331-345aa | SEAPPFFVLHGEKDP | 56 |
| | LipR (Rv3084) | R3 | 121-135aa | EEMAAVYTRLLDDGl | 57 |
| | Rv2350c(PLCB) | S7 | 261-275aa | FKQAADPRSNLARFG | 58 |
| | Rv2350c(PLCB) | S3 | 91-105aa | DPAGVTLPYRFDTTR | 59 |
| | Rv2350c(PLCB) | S8 | 281-295aa | PLDFAADVRNNRLPK | 60 |
| | Rv2350c (PLCB) | S4 | 101-115aa | FDTTRGPLVAGECVN | 61 |
| | LipT (Rv2045c) | T4 | 431-445aa | IYRTRFGALLTAAAD | 62 |
| | LipT (Rv2045c) | T2 | 31-45aa | DGVHRWRSIPYARAP | 63 |
| | LipT (Rv2045c) | 17 | 461-475aa | RCFSQIGVPGDDWPA | 64 |
| | LipU (Rv1076) | U1 | 71-853a | GGAFLTCGANSHGRL | 65 |
| | Rv1755c (PLCD) | X6 | 271-280aa | PTRGIPSGPC | 66 |
| | LipY (Rv3097c) | Y1 | 221-235aa | SVVQITPAHPTGEYV | 67 |
| bad | Rv0183 | B12 | 161-175aa | FAYGVERPDNYDLMV | 68 |
| | Rv0183 | B10 | 131-145aa | DFDTLVGIATREYPG | 69 |
| | Rv0183 | B8 | 81-95aa | HGLGEHARRYDHVAQ | 70 |
| | Rv0183 | B14 | 241-255aa | GRALLQVGETMPRRA | 71 |
| | Rv0183 | B7 | 11-25aa | SAGSRPEFSASTLTS | 72 |
| | Rv0183 | B17 | 301-315aa | EVFNEPERNQVLDDV | 73 |
| | Rv0183 | B11 | 141-155aa | REYPGCKRiVLGHSM | 74 |
| | Rv0183 | B2 | 61-75aa | RIVYDVWTPDTAPQA | 75 |
| | Rv0183 | B13 | 211-225aa | DFTAISRDPEVVQAY | 76 |
| | Rv0183 | B9 | 121-135aa | LVRDISEYTADFDTL | 77 |
| | Rv0183 | B5 | 261-275aa | PLLVLHGTDDRLIPI | 78 |
| | Rv0183 | B3 | 101-115aa | GLVTYALDHRGHGRS | 79 |
| | Rv0183 | B4 | 111-125aa | GHGRSGGKRVLVRDI | 80 |
| | LipC (Rv0220) | C22 | 341-355aa | QARSFVERLRAVSRS | 81 |
| | LipC (Rv0220) | C15 | 81-95aa | RRRDRASTQEVSVAA | 82 |
| | LipC (Rv0220) | C13 | 11-25aa | GVAYIRWLLRARPAD | 83 |
| | LipC (Rv0220) | C8 | 251-265aa | NDPQYQAELPEGSDT | 84 |
| | LipC (Rv0220) | C16 | 131-145aa | RRVLYGDDPAQLLDV | 85 |
| | LipC (Rv0220) | C9 | 271-285aa | VGIYGRYDWEDRSTP | 86 |
| | LipC (Rv0220) | C11 | 311-325aa | RDASPIQRVTRNAPP | 87 |
| | LipC (Rv0220) | C17 | 201-215aa | HRWPRHILDVKTAIA | 88 |
| | LipC (Rv0220) | C23 | 351-365aa | AVSRSQVGYLELPGA | 89 |
| | LipC (Rv0220) | C25 | 371-385aa | LLDGARTGPTAHAIA | 90 |
| | Rv3452 | D6 | 18M95aa | PQFGSKTINLCNNGD | 91 |
| | Rv3452 | D2 | 51-65aa | EVVFARGTGEPPGLG | 92 |
| | Rv3452 | D8 | 41-55aa | PPASAGCPDAEVVFA | 93 |
| | Rv3452 | D3 | 71-85aa | FVSSLRQQTNKSIGT | 94 |
| | Rv3452 | D10 | 151-165aa | LPPAADDHIAAIALF | 95 |
| | Rv3452 | D4 | 101-115aa | DGANDASDHIQQMAS | 96 |
| | Rv1984 | E3 | 81-95aa | ASDDYRASASNGSDD | 97 |
| | Rv1984 | E4 | 21-35aa | VSAPAGGRAAHADPC | 98 |
| | Rv1984 | E6 | 51-65aa | GLGDVGEAFVDSLTS | 99 |
| | Rv1984 | E7 | 71-85aa | SIGVYAVNYPASDDY | 100 |
| | LipF (Rv3487c) | F13 | 151-165aa | VRAAAAKNMVDGRPE | 101 |
| | LipF (Rv3487c) | F11 | 101-115aa | QRLQCDDEKPAAIVA | 102 |
| | LipF (Rv3487c) | F4 | 241-255aa | FIRDATADSSLSPVH | 103 |
| | LipF (Rv3487c) | F1 | 71-85aa | YQWLRARGYRPEQIV | 104 |
| | LipF (Rv3487c) | F2 | 161-175aa | DGRPEDLYEPLDHIE | 105 |
| | LipF (Rv3487c) | F5 | 251-265aa | LSPVHRSRYVAGSPR | 106 |
| | LipF (Rv3487c) | F14 | 231-245aa | ATR5LRQIGQFIRDA | 107 |
| | LipF (Rv3487c) | F8 | 31-45aa | SHSRIVNALSGFAES | 108 |
| | LipF (Rv3487c) | F10 | 81-95aa | PEQIVLAGDSAGGYL | 109 |
| | LipF (Rv3487c) | F3 | 171-185aa | LDHIESSLPPTLIHV | 110 |
| | LipF (Rv3487c) | F6 | 261-277aa | AGSPRAASRGAFGQSPI | 111 |
| | LipF (Rv3487c) | F9 | 61-75aa | GMALDDCHDAYQWLR | 112 |
| | LipG (Rv0646c) | G5 | 171-185aa | PPDSPRDVIVDNAVR | 113 |
| | LipG (Rv0646c) | G2 | 11-25aa | GDVKLYYEDMGDLDH | 114 |
| | LipG (Rv0646c) | G4 | 61-75aa | NRDVGLSTKTERHRP | 115 |
| | LipG (Rv0646c) | G7 | 291-301aa | GELTRNFSEAG | 116 |
| | LipG (Rv0646c) | G11 | 191-205aa | GSPAYPIPEDQVRAE | 117 |
| | LipG (Rv0646c) | G1 | 1-15aa | VDRISGTAVSGDVKL | 118 |
| | LipG (Rv0646c) | G10 | 18M95aa | DNAVRVSKIIGSPAY | 119 |
| | LipG (Rv0646c) | G8 | 71-85aa | ERHRPGQPLATRLVR | 120 |
| | LipG (Rv0646c) | G6 | 281-295aa | LPRQLWDRVIGELTR | 121 |
| | LipH (Rv1399c) | H1 | 41-55aa | QLKTPPELLPELRIE | 122 |
| | LipI (Rv1400c) | 13 | 41-55aa | RLRDLPRQPVHPELR | 123 |
| | LipI (Rv1400c) | 12 | 31-45aa | IDDGIEAVRQRLRDL | 124 |
| | Rv3451 | J3 | 91-105aa | RLQLHGGDGANDAIS | 125 |
| | Rv3451 | J2 | 41-55aa | ADGCPDAEVTFARGT | 126 |
| | Rv3451 | J5 | 191-205aa | TDPICHVGPGNEFSG | 127 |
| | Rv3451 | J1 | 1115aa | VNNRPIRLLTSGRAG | 128 |
| | Rv3451 | J4 | 161-175aa | VFGNPSNRAGGSLSS | 129 |
| | Rv3451 | J7 | 251-262aa | TAAPAPESLHGR | 130 |
| | Rv3451 | J6 | 221-235aa | FWQRLRAGSVPHLP | 131 |
| | Rv3802c | K6 | 131-145aa | HNPLTTDNQMSYNDS | 132 |
| | Rv3802c | K9 | 251-265aa | QGDLICAAPAQAFSP | 133 |
| | Rv3802c | K8 | 211-225aa | RQQGVFNQVPPSPRG | 134 |
| | Rv3802c | K4 | 61-75aa | HKPRPAFQDASCPDV | 135 |
| | Rv3802c | K2 | 31-45aa | AVVIMLRGAESPPSA | 136 |
| | Rv3802c | K7 | 181-195aa | AGDVASDIGNGRGPV | 137 |
| | Rv3802c | K1 | 1-15aa | MAKNSRRKRHRILAW | 138 |
| | Rv3802c | K3 | 51-65aa | LPPGTPAHPHKPRP | 139 |
| | LipM (Rv2284) | M2 | 121-135aa | SAGLWRRPAGGGTAK | 140 |
| | LipM (Rv2284) | M4 | 141-155aa | RMLRIYRDYAHDGDI | 141 |
| | LipM (Rv2284) | M5 | 271-285aa | ALTPNDPRFQPGFEE | 142 |
| | LipM (Rv2284) | M1 | 111-125aa | SGLGPDRRTASAGLW | 143 |
| | LipN (Rv2970c) | N3 | 281-295aa | YLRDSDVDPADPRLS | 144 |
| | LipN (Rv2970c) | N2 | 271-285aa | KRDIDWFHTQYLRDS | 145 |
| | LipN (Rv2970c) | N1 | 111-125aa | DLSIPGPAGEIPARH | 146 |
| | LipO (Rv1426c) | O5 | 211-225aa | VCVSLNYRVSPRHTW | 147 |
| | LipO (Rv1426c) | O8 | 321-335aa | KRKFSTHRDIFVDAS | 148 |
| | LipO (Rv1426c) | O4 | 161-175aa | ANLADIWRRRDLPRD | 149 |
| | LipO (Rv1426c) | O2 | 61-75aa | ALRRGRRGDFGGLKG | 150 |
| | LipO (Rv1426c) | O1 | 1-15aa | MRFRRMARPRPLTRA | 151 |
| | Rv2351c(PLCA) | P8 | 411-425aa | SQLKLIRARFGVPVP | 152 |
| | Rv2351c(PLCA) | P1 | 1-15aa | MSRREFLTKLTGAGA | 153 |
| | Rv2351c(PLCA) | P7 | 351-365aa | TPPTAPPGTPGEFVT | 154 |
| | Rv2351c(PLCA) | P5 | 251-265aa | NNGLVQAFRQAADPR | 155 |
| | Rv2351c(PLCA) | P4 | 221-235aa | IMPENLEDAGVSWKV | 156 |
| | LipQ (Rv2485c) | Q2 | 131-145aa | GPHRRYAAQTSDIPY | 157 |
| | LipQ (Rv2485c) | Q1 | 101-115aa | PDFRDLVWHPTGEQS | 158 |
| | LipQ (Rv2485c) | Q7 | 261-275aa | SANDPALQPGFESAD | 159 |
| | LipQ (Rv2485c) | Q3 | 141-155aa | SDIPYGPGGRENLLD | 160 |
| | LipQ (Rv2485c) | Q5 | 161-175aa | DLAPGRRAPVLIQVP | 161 |
| | LipR (Rv3084) | R1 | 1-15aa | MNLRKNVIRSVLRGA | 162 |
| | LipR (Rv3084) | R5 | 241-255aa | ICVDADKIETACAAS | 163 |
| | LipR (Rv3084) | R2 | 41-55aa | RAPKGTRFQRVSIAG | 164 |
| | LipR (Rv3084) | R7 | 291-308aa | RLRGHLHQSQGQPRGWK | 165 |
| | LipR (Rv3084) | R4 | 131-145aa | LDDGLDPKTTVIAGD | 166 |
| | LipR (Rv3084) | R6 | 251-265aa | ACAASKTSIEHRRFA | 167 |
| | Rv2350c(PLCB) | S5 | 221-235aa | SWRIMPENLEDAGVS | 168 |
| | Rv2350c(PLCB) | S10 | 401-415aa | RGPLMVHDTFDHTST | 169 |
| | Rv2350c(PLCB) | S12 | 491-505aa | PFPQSMPTQETAPTR | 170 |
| | Rv2350c(PLCB) | S6 | 251-265aa | VVGYNGLVNDFKQAA | 171 |
| | Rv2350c(PLCB) | S9 | 361-375aa | GTPGEFVTVPDIDSV | 172 |
| | Rv2350c(PLCB) | S2 | 51-65aa | QENRSFDHYFGTLSD | 173 |
| | Rv2350c(PLCB) | S1 | 41-55aa | TDIEHIVLLMQENRS | 174 |
| | Rv2350c(PLCB) | S11 | 451-465aa | PNPSKPNLDHPRLNA | 175 |
| | LipT (Rv2045c) | I5 | 441-455aa | TAAADRRAALRVSNE | 176 |
| | LipT (Rv2045c) | T1 | 1-15aa | VALESATVGSMHERT | 177 |
| | LipT (Rv2045c) | T3 | 401-415aa | YLYRYDYAPRTLRWS | 178 |
| | LipT (Rv2045c) | T10 | 491-505aa | RIEFDPHQHRRIAWD | 179 |
| | LipT (Rv2045c) | T8 | 471-485aa | DDWPATTQDDRAVLV | 180 |
| | LipU (Rv1076) | U3 | 281-297aa | AIRSLRQIGEYIREATG | 181 |
| | LipU (Rv1076) | U2 | 201-215aa | VASAAARNQVDGEPE | 182 |
| | Rv2349c(PLCC) | V4 | 251-265aa | RNGYVGSFKQAADPR | 183 |
| | Rv2349c(PLCC) | V7 | 401-415aa | GLMVHDRFDHTSQLQ | 184 |
| | Rv2349c(PLCC) | V1 | 71-853a | TPTPLFQQKGWNPET | 185 |
| | Rv2349c(PLCC) | V6 | 361-375aa | GEWIPNSVDIDKVDG | 186 |
| | Rv2349c(PLCC) | V2 | 191-205aa | ISATVNPDGDQGGPQ | 187 |
| | Rv2349c(PLCC) | V8 | 451-465aa | PSPPNLDHPVRQLPK | 188 |
| | Rv2349c(PLCC) | V3 | 241-255aa | LGGLNDTSLSRNGYV | 189 |
| | LipW (Rv0217c) | W1 | 41-S5aa | MSRTPPDIEVLTLES | 190 |
| | LipW (Rv0217c) | W6 | 191-205aa | PHRYLWNGRANRFGW | 191 |
| | LipW (Rv0217c) | W2 | 51-65aa | LTLESGVGVRLYRPA | 192 |
| | LipW (Rv0217c) | W4 | 91-105aa | DRLCLRFSSRLGITV | 193 |
| | Rv1755c(PLCD) | X4 | 251-265aa | NRGIPYRVPDPQIMP | 194 |
| | Rv1755c(PLCD) | X1 | 131-145aa | TPGEYVTVPDIDQVP | 195 |
| | Rv1755c(PLCD) | X3 | 171-185aa | GPQMVHDTFDHTSQL | 196 |
| | Rv1755c(PLCD) | X5 | 261-275aa | PQIMPTGQETTPTRGI | 197 |
| | Rv1755c(PLCD) | X2 | 141-155aa | IDQVPGSGGIRGPIG | 198 |
| | Rv2301 | Z2 | 61-75aa | ALRSKVNKNVGVYAV | 199 |
| | Rv2301 | Z4 | 171-185aa | IELCHGDDPVCHPAD | 200 |

In order to determine the immunogenicity of the peptides, they are tested using the following ELISA test:

The peptides are fixed on a Maxisorp (high binding) plate: Incubation overnight at 4° C., concentration of the protein in μg/ml from 20 to 50.

The wells are rinsed with 300 μL of phosphate buffered saline (PBS).

The “wells” are blocked for 2 hours at ambient temperature (19° C.-25° C.) with 200 μL of bovine serum albumin (BSA) 2% and 0.01% of Tween 20.

The wells are rinsed with 200 μL of PBS.

One deposits 100 μL of serums from patients suffering from active TA diluted at 1/100^{th} in blocking solution and incubates for one hour at 37° C.

Perform 3 washes (300 μL) PBS-Tween 20 (0.05%).

One adds 100 μL of antibodies conjugated with peroxidase, diluted at 1/20,000 with blocking solution and incubates for 1 h at 37° C.

Perform 3 washes (300 μL) PBS-Tween 20 (0.05%).

One adds 100 μL of 3,3′,5,5′-tetramethylbenzidine (TMB) and incubates for 20 minutes in the dark.

One deposits 50 μL of sulfuric acid at 1N.

A spectrophotometer is used at 450 nm to read the quantity of TMB converted by the peroxidase (indicative of the formation of an immune complex between the peptides and the antibodies contained in the serum).

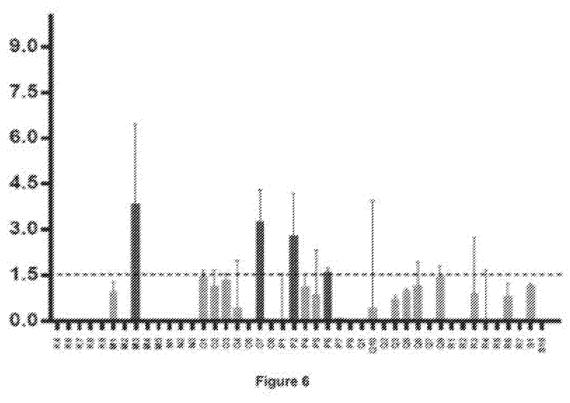

FIGS. 4 to 7 show histograms showing the ratio R (Index) of the different tested peptides.

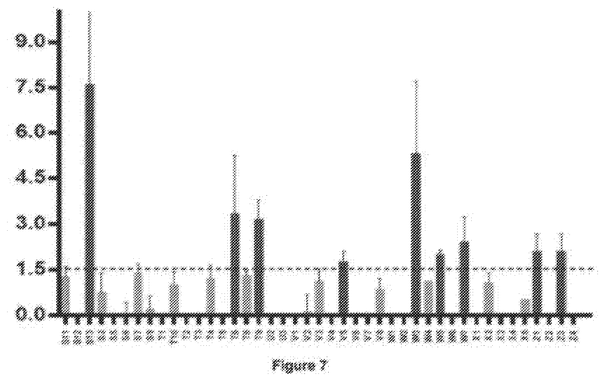

FIG. 8 shows the reactivity of the most promising peptides C2, C12, M3, V5 and G9 (respectively shown by sequences SEQ ID NO: 1 to 5).

### Example 2—Reactivity of 4 Peptides on Samples from Different Individual Patients from Pools Used for the Screening

In order to confirm that the sectioned peptides are indeed capable of diagnosing active tuberculosis, the inventors tested the reactivity of 4 peptides (M3: SEQ ID NO: 3; C12: SEQ ID NO: 2; C2: SEQ ID NO: 1 and 07: SEQ ID NO: 6).

The following table shows the results obtained with the serum of 16 patients:

| Patient | M3 | C12 | C2 | O7 |
|---|---|---|---|---|
| #1 | +++ | +++ | +++ | +++ |
| #2 | +++ | +++ | + | +/− |
| #3 | +++ | +/− | +/− | +/− |
| #4 | +++ | +++ | +/− | + |
| #5 | +++ | + | + | +/− |
| #6 | +++ | + | +/− | +++ |
| #7 | +++ | +++ | +/− | +/− |
| #8 | +++ | +/− | +++ | +++ |
| #9 | +++ | +++ | +/− | − |
| #10 | +++ | +++ | +++ | +++ |
| #11 | + | +/− | +/− | − |
| #12 | + | +/− | +/− | +/− |
| #13 | + | + | +/− | − |
| #14 | +/− | + | + | + |
| #15 | +/− | − | +/− | − |
| #16 | − | +++ | − | − |

+++: extremely positive;
+: positive;
+/−: limited positive value;
−: negative.

The results show that among the best 5 identified candidates, 3 (M3, C2, C12) detect the large majority of tested individuals suffering from active TB (or 94%), confirming the results obtained with the pools of patients. Conversely, the diagnostic performance of O7 is lower with less than 70% sensitivity for the identification of cases of active TB on these tested patients.

### Example 3—Comparison of the Reactivity of the Peptides According to the Number of Pools of Samples Used

With the aim of evaluating the reactivity of the peptides and selecting the most promising for the active Tuberculosis diagnostic test, the inventors evaluated the immunogenicity of each of the peptides on several different pools of samples. Those with a reactivity for all of the tested pools were selected as the best candidates. The table below shows an example of 3 tested peptides with two different pools of positive samples.

| | OD | Ratio | Ratio |
|---|---|---|---|
| Peptides | Negative pool | Positive pool 1 | Positive pool 2 |
| D7 | 0.303 | 1.7 | 1.7 |
| C4 | 0.077 | −0.3 | 1.8 |
| B4 | 0.408 | 0.9 | 1.0 |

The peptides for which a ratio of 1.5 was found were selected at the end of the screening and are among the best diagnostic candidates for active TB. Among all of the tested peptides, some have a ratio >1.5 for both pools of patients (example D7), while others are positive for one of the two pools (example C4) or are negative for both pools (example B4). The screening of the peptides was next refined by using samples of individual patients who were or were not infected by active TB.

### Example 3—Alternative Method for Detecting Tuberculosis

Following the ELISA proof of concept done by the inventors, lateral flow tests were developed by integrating the best Tuberculosis diagnostic candidate peptides described in the invention. These results were not good enough to satisfy the expectations of the specifications and the targeted product profile of the WHO. The inventors therefore preferred to set aside this lateral flow strategy to look for a better performing alternative.

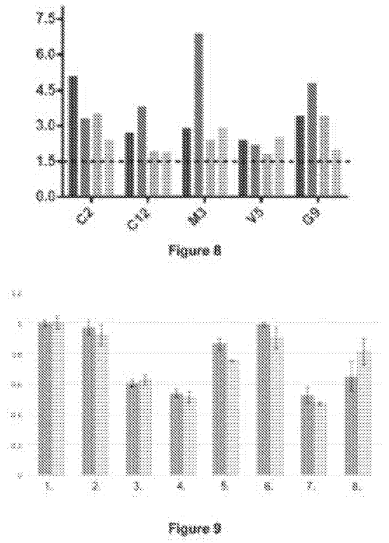

The inventors developed a technology on magnetic nanoshells, making it possible to significantly improve the performance of the ELISA test, without washes and in just a few minutes. This strategy is relevant with the perspective of miniaturizing the current existing elements to propose a rapid laboratory diagnostic solution, as well as a future Point-of-Care test. The nanoshell technology was developed on a lab table version for the laboratory, not able to be taken off-site. This is the initial version of the test. The first tests done with this version show that by combining the magnetic nanoshells with the best diagnostic candidate peptides of the invention, and using a strategy for detecting antibodies in patients' serum, the inventors were obtaining results at least as good as the results of ELISA, but with a smaller quantity of peptides, and above all in less than 10 minutes (versus approximately two hours for a traditional ELISA). An example of results for one of the best 5 peptides is illustrated in FIG. 9.

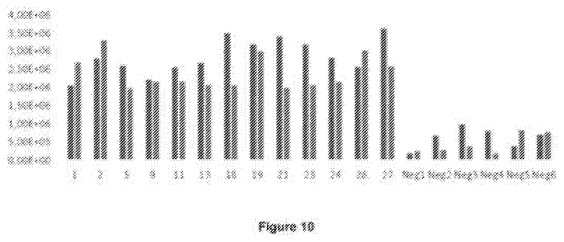

The inventors therefore pushed the experiments, and after optimization tests (dilution of the sera, test of the peptides in combination, choice of the detection antibody, background noise control, etc.), the inventors identified the best peptide to be used from among the best 5 candidates. In parallel, a transportable prototype was developed and the inventors tested 19 samples (13 patients with active TB and 6 who were negative for the disease). The results of FIG. 10 show that this transportable prototype makes it possible to obtain results at least as good as those obtained with the lab table system previously developed by the inventors, thus allowing a clear discrimination between positive patients and negative patients,

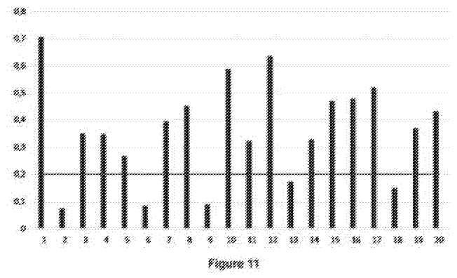

Lastly, the most recent tests done on 20 other samples evaluated, in single blind, the ability of the transportable prototype to discriminate between patients with active TB and those who are not infected (FIG. 11), confirming the performance of the test integrating the combination of diagnostic candidate peptides and the technology on nanoshells in its transportable version.

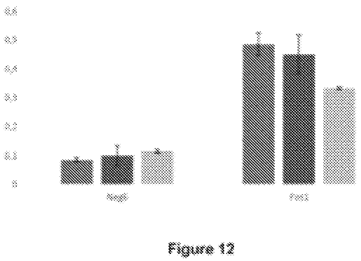

Reproducibility tests were also conducted and do not show any variation. The stability of the nanoshell-peptide coupling can be improved, since a gradual decrease of the test signal over time was observed (FIG. 12).

The transportable prototype therefore shows very encouraging results.

The invention is not limited to the embodiments that have been shown, and other embodiments will clearly appear to one skilled in the art.

1. An in vitro method of screening at least one immunogenic peptide of interest capable of recognizing at least one antibody originating from the serum of individuals suffering from active tuberculosis, said at least one immunogenic peptide being a hydrophilic peptide originating from a hydrophobic protein, said hydrophobic protein being a wall protein, or secreted from bacteria from the Mycobacterium genus, said hydrophobic protein having a lipolytic activity,
said method comprising the following steps:
bringing into contact of at least one hydrophilic peptide originating from at least one hydrophobic peptide with
successively at least two independent pools of serums originating from patients suffering from confirmed active tuberculosis, to allow the formation of immune complexes between said antibodies and said peptides to be screened, and
at least one control sample originating from an individual not suffering from tuberculosis,
detecting the formation of immune complexes in the previous step,
conducting a first selection of the peptides of interest for which the value of a ratio R is greater than or equal to 1.5 for at least one of the pools of independent serums originating from patients suffering from confirmed active tuberculosis,
the ratio R being the normalized measurement value of the formation of immune complexes to the normalized measurement value obtained from the sample originating from a healthy individual.

2. The screening method according to claim 1, further including the following steps:
bringing the peptides selected in the first selection step into contact with each of the individual serums making up said independent pools of serums originating from patients suffering from confirmed active tuberculosis, to allow the formation of immune complexes between said antibodies and said peptides to be screened,
detecting the formation of immune complexes in the preceding step, and
carrying out a second selection of peptides of interest for which the value of the ratio R is greater than or equal to 1.5 for with each of the individual serums making up said independent pools of serums for patients with confirmed active tuberculosis.

3. The method according to claim 1, wherein during the first selection, only the peptides for which the value of a ratio R is greater than or equal to 1.5 for at least two of the independent pools of serums coming from patients suffering from confirmed active tuberculosis are selected.

4. The method according to claim 1, wherein the hydrophilic peptides have a size from 15 to 25 amino acids.

5. A hydrophilic peptide, comprising, or consisting of, 15 to 25 amino acids, originating from a hydrophobic protein, said hydrophobic protein being a wall protein or secreted from bacteria of the Mycobacterium genus, said hydrophobic protein having a lipolytic activity.

6. The hydrophilic peptide according to claim 6, said peptide being represented by any one of the following sequences: SEQ ID NO: 1 to SEQ ID NO. 30, in particular by any one of the following sequences: SEQ ID NO: 1 to SEQ ID NO: 5.

7. An in vitro method of diagnosing an individual who may be suffering from active tuberculosis, said method including:
a step of bringing a blood sample from said individual into contact with at least one hydrophilic peptide according to claim 5, and
a step of detecting an immune complex between at least one antibody of said blood sample and said peptides.

8. A kit for diagnosing active tuberculosis, including:
at least one hydrophilic peptide as defined in any one of claims 5, and
means for identifying immune complexes between at least one antibody originating from a blood sample of an individual and said at least one peptide.

9. The kit according to claim 8, including means for identifying immune complexes between at least one antibody originating from a blood sample of an individual are arranged on a chromatographic-type substrate.

10. The kit according to claim 8, wherein said at least one hydrophilic peptide is coupled with magnetic nanoshells.

11. (canceled)
